(19) United States
(12) Patent Application Publication
(10) Pub. No.: US 2019/0237166 A1
(43) Pub. Date: Aug. 1, 2019

(54) METHOD FOR OPTIMIZING FLUORESCENCE-BASED DETECTION

(71) Applicant: THE ROYAL INSTITUTION FOR THE ADVANCEMENT OF LEARNING/MCGILL UNIVERSITY, Montreal (CA)

(72) Inventors: David JUNCKER, Verdun (CA); Milad DAGHER, Montreal (CA); Michael KLEINMAN, Montreal (CA)

(21) Appl. No.: 16/153,071

(22) Filed: Oct. 5, 2018

Related U.S. Application Data
(60) Provisional application No. 62/568,998, filed on Oct. 6, 2017.

(51) Int. Cl. G16C 20/70; G01N 33/542; G16C 20/80; G01N 21/64

(52) U.S. Cl. CPC G16C 20/70; G01N 33/542; G01N 2021/6441; G01N 21/6428; G01N 21/6456; G16C 20/80

(57) ABSTRACT

Systems and methods for optimizing detection of light-emissive components of a multi-fluorescence spectra. The method comprises obtaining a multi-fluorescence based spectra of a plurality of light-emissive components and determining a model of ensemble multi-fluorescence of said light-emissive components that are stochastically distributed.

### CROSS-REFERENCE TO RELATED APPLICATION

This application claims the benefit of and priority to U.S. provisional patent application No. 62/568,998, filed on Oct. 6, 2017, the content of which is herein incorporated in entirety by reference.

### FIELD OF TECHNOLOGY

The present disclosure relates to systems for optimizing fluorescence-based detection. The present technology also relates to methods for optimizing fluorescence-based detection. The present technology further relates to apparatuses for performing methods for optimizing fluorescence-based detection.

### BACKGROUND INFORMATION

Barcoded microparticles (BMPs) are paramount for multiplexed suspension assays as they allow distinguishing probes from a large mixture. Fluorescent encoding of BMPs using precise proportions of multicolor classifier dyes is the most popular approach as it allows simple and high throughput read-out by flow cytometry. In an ideal BMP system, the concentrations and fluorescence intensities of differently-colored classifier dyes are orthogonal and may be independently controlled to allow straightforward encoding and decoding. However, spectral overlap between common dyes, such as organic fluorophores and quantum dots, becomes unavoidable beyond 2 or 3 colors because of the limited spectral bandwidth available (˜350-750 nm), giving rise to multicolor Förster resonance energy transfer (mFRET) and cascades thereof. As a result, efforts to expand the barcoding capacity are met with rapidly intensifying mFRET and an intractable ensemble fluorescence, imposing labour-intensive experimental iterations to obtain distinguishable barcode responses and barring fully-automated decoding. In addition, common microparticle (MP) functionalization methods are sensitive to competing physical and chemical properties of the different dyes and result in poor control over dye proportions.

In recent years, there has been a growing interest in exploiting mFRET for tracking multiple intermolecular distances and assembling energy-harvesting photonic networks. However, mFRET models have been restricted to single molecules with fixed inter-dye distances. An analytical model describing mFRET between stochastically distributed dyes, as is the case for BMPs, is still lacking. FRET (mFRET), which arises between multiple, stochastically distributed fluorophores, lacks a mechanistic model and remains intractable. mFRET notably arises in fluorescently barcoded microparticles, resulting in a complex, non-orthogonal fluorescence response that impedes their encoding and decoding.

There is thus still a need to be provided with an analytic model describing mFRET between stochastically distributed dyes, as is the case for BMPs, and which could allow for barcoding at extreme FRET levels.

### SUMMARY OF TECHNOLOGY

According to various aspects, the present technology relates to a method for optimizing detection of a plurality of light-emissive components from a multi-fluorescence spectra, the method being executable by a processor of a computer system operatively communicating with an imaging device, the method comprising: a) obtaining a multi-fluorescence based spectra of at least some of the light-emissive components; b) determining a model of ensemble multi-fluorescence of the light-emissive components and of the imaging device, wherein the light-emissive components are stochastically distributed; and c) determining proportion of each light-emissive component of the multi-fluorescence based spectra of a) based on the model of b).

According to various aspects, the present technology relates to a method for calibrating a multicolor fluorescence model of a multitude of light-emissive components and an imaging device, the method being executable by a processor of a computer system operatively communicating with the imaging device, the method comprising: a) obtaining a first fluorescence information about the individual light-emissive components using the imaging device, b) obtaining a second fluorescence information about at least some pairs of light-emissive components using the imaging device, c) determining the constants of the multicolor fluorescence model using the first and second fluorescent information obtained in a) and b); wherein the constants obtained in c) account for the non-linearity in the multicolor fluorescence model.

According to various aspects, the present technology relates to a kit for calibrating a multi-fluorescence model of a multitude of light-emissive components and an imaging device, the kit comprising: a) a first set of particles labeled with light-emissive components; and b) a second set of particles labeled with at least some pairs of the light-emissive components. In some embodiments, the kit further comprises instructions on how to use the kit in the calibration of a multi-fluorescence model of a multitude of light-emissive components and of an imaging device.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Further aspects and advantages of the present technology will become better understood with reference to the description in association with the following in which:

FIG. 1 is a flowchart illustrating an example method for optimizing detection of light-emissive components, according to certain embodiments of the present technology.

FIG. 2 is a schematic illustration of an example system for optimizing detection of light-emissive components, according to certain embodiments of the present technology.

FIGS. 3A-3D illustrate spectrally overlapping dyes and the impact of mFRET on BMP response. FIG. 3A: normalized absorption and emission (abs/em) spectra of the four classifier dyes; FIG. 3B: schematic representation of MP readout by flow cytometry; FIG. 3C: fluorophore photophysical properties, excitation/readout optics and calculated inter-dye Förster radii (Rda); and FIG. 3D: effect of spectral overlap on the relative positions of BMP clusters given by their dye proportions (σ1, σ2, σ3, σ4) within intensity spaces 11-12.

FIGS. 4A-4C illustrate one-pot DNA-assisted microparticle labelling conserving dye proportions. FIG. 4A: one-pot reaction of streptavidin-coated microparticles; FIG. 4B: coefficient of validation (CV) of the fluorescence intensity; and FIG. 4C median fluorescence intensity (MFI).

FIGS. 5A-5F illustrate emFRET model and experimental validation of multi fluorescence model (MFM). FIG. 5A: schematic representation of emFRET model and its conversion to e2FRET configuration; FIG. 5B: input amount of LO_{f} (n_{f}; FIG. 5C: emFRET-predicted total FRET efficiency for dye f (E^{T}_{f}), for four color barcodes; FIGS. 5D-5F: a comparison of measurements.

FIGS. 6A-6E illustrate in silico design and experimental verification of high density four-color barcodes with extreme emFRET, showing in FIG. 6A are 6D plots of the MFM-designed positions of 580 barcodes in 4D intensity-space (i₁, i₂, i₃, i₄). FIG. 6B shows the breakdown of inter-dye FRET efficiencies between all dye combinations for each barcode showcasing the extreme levels of FRET in some cases. FIGS. 6C, 6D and 6E show the experimental intensity scatter plots of BMPs are overlaid with their MFM-predicted values from the three sub-plots highlighted in FIG. 6A to showcase the excellent agreement with the MFM, save some for barcodes with high n3 and n4 in FIG. 6C.

FIGS. 7A-7D illustrate the fully automated BMP decoding using an MFM-initialized GMM. FIGS. 7A-7B: fraction (in %) of BMPs classified to the correct cluster with respect to the posterior probability thresholds using a GMM algorithm (FIG. 7A) without and (FIG. 7B) with initial conditions based on MFM-predicted intensities. FIGS. 7C-7D heatmaps quantifying the fraction (in %) of BMPs from barcode x (actual) that were assigned to barcode y using our 4D GMM-based decoding algorithm, performed (FIG. 7C) without and (FIG. 7D) with MFM-predicted intensities as initial conditions.

FIG. 8 is a flowchart illustrating an example method for determining mFRET between stochastically distributed dyes.

FIG. 9 is a block diagram illustrating an example computer system for implementing the method of FIG. 5.

FIGS. 10A-10C illustrate the parameters within the MFM equations determined using 18 barcodes via the process flow described in FIG. 10A. FIGS. 10B and 10C show fitting of the one-color BMPs by linear regression to calculate the (FIG. 10B) direct excitation and (FIG. 10C) bleed-through.

It is to be expressly understood that the description and drawings are only for the purpose of illustrating certain embodiments of the present technology and are an aid for understanding. They are not intended to be a definition of the limits of the technology.

### DETAILED DESCRIPTION

Before continuing to describe the present disclosure in further detail, it is to be understood that this disclosure is not limited to specific devices, systems, methods, or uses or process steps, and as such they may vary. It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, “detect” means determining one or more of: a presence of absence of one or more light-emissive components, a proportion of one or more light emissive components, and a concentration of one or more light emissive components. The term encompasses qualitative, semi-quantitative, and quantitative determinations. In embodiments where the light-emissive components are associated, such as through labelling, with a substance to be detected, such as an analyte, “detect” may mean the presence or absence of the analyte such as an oligonucleotide and encompasses qualitative, semi-quantitative, and quantitative determinations. A quantitative determination gives a numerical value for the mass or molar quantity of the analyte, which will generally be subject to some degree of uncertainty due to typical sources of error. Molar quantity refers to the number of molecules, whether expressed as a literal number of molecules (e.g., 10¹⁴ molecules) or as a number or fraction of moles (e.g., 1 nanomole). A semi-quantitative determination gives at least an indication of the relative amount of the analyte, such as whether it is lower, approximately equal to, or higher than a threshold value or reference sample. In some embodiments, approximately equal to a value means within an order of magnitude. In some embodiments, approximately equal to means within or equal to five-fold. In some embodiments, approximately equal to means within or equal to two-fold. In some embodiments, approximately equal to means within or equal to 50%. In some embodiments, approximately equal to means within or equal to 34%. In some embodiments, approximately equal to means within or equal to 25%. In some embodiments, approximately equal to means within or equal to 20%. In some embodiments, approximately equal to means within or equal to 15%. In some embodiments, approximately equal to means within or equal to 10%. In some embodiments, approximately equal to means within or equal to 5%.

As used herein, the term “quantify” means determining the amount of an analyte, such as an oligonucleotide, and encompasses semi-quantitative and quantitative determinations. As used herein, the term “determining an amount” means a quantitative determination.

As used herein, the term “light” generally refers to electromagnetic radiation, having any suitable wavelength (or equivalently, frequency). For instance, in some embodiments, the light may include wavelengths in the optical or visual range (for example, having a wavelength of between about 400 nm and about 700 nm, i.e., “visible light”), infrared wavelengths (for example, having a wavelength of between about 300 micrometers and 700 nm), ultraviolet wavelengths (for example, having a wavelength of between about 400 nm and about 10 nm), or the like. In certain cases, as discussed in detail below, more than one entity may be used, i.e., entities that are chemically different or distinct, for example, structurally. However, in other cases, the entities may be chemically identical or at least substantially chemically identical.

As used herein, the terms “light-emissive components”, “dyes”, and “fluorophores” are used interchangeably.

The term “bead” as used herein encompasses “particles” (e.g., microparticles, nanoparticles) and refers to a solid particle having a globular or roughly spherical shape, which may be porous or non-porous. Non-porous surfaces may be present to increase surface area thus allowing for the association of increased number of surface bound molecules as compared to, for example, “smooth” surfaces.

As used herein, the term “substrate” refers to any component or substance on which or onto which the light-emissive component as defined herein may be attached. Examples of substrates include, but are not limited to: cells, molecules, nucleic acid molecules, amino acid molecules, peptides, polypeptides, proteins, carbohydrates, lipids, chemicals, drugs, or the like. A person skilled in the art will readily appreciate that, in come instances, the substrates may be labelled with the light-emissive components of the present technology.

As used herein, the term “quantitating” refers to the act of determining the amount or proportion of a substance in a sample.

The present technology steams from the discoverers's elucidation of a Förster resonance energy transfer (FRET) model, in particular of an ensemble multicolour FRET (emFRET) model and its incorporation within a multicolor fluorescence model (MFM) (e.g., multi fluorescence spectra). Through calibration of the MFM with the specific imaging device (e.g. a flow cytometer) and the encoding method (e.g. surface-labeled microparticles), the physical constants pertaining to the optical system and the encoding method can be calibrated, completing the parameterization of the MFM and enabling several applications. To establish the model, a labelling method employing DNA as a homogeneous crosslinker allows precise control over the dye proportions on BMPs. This proportional labeling allows for great simplification of the model and allows extracting the physical constants pertaining to the system.

In one embodiment, the emFRET model affords quantitative insight into stochastic mFRET cascades, allowing rational design and optimizing and/or fine-tuning of the spectral response (Milad Dagher et al., Nature Nanotechnology, 13, 925-932, 2018, incorporated herein by reference).

In one embodiment, the approach enables the use of spectrally overlapping light-emissive components for high-capacity barcoding by extending barcoding into extreme FRET regimes and allows for accurate in silico barcode design and automatic readout by, for example, flow cytometry.

In one embodiment, the present technology thus relates to methods, processes and systems for optimizing detection of a multi-fluorescence based spectra. In some instances, the multi-fluorescence based spectra comprises fluorescence emitted by a plurality of light-emissive components.

In one embodiment, the present technology relates to methods, processes and systems for optimizing detection of light-emissive components of a multi-fluorescence spectra. In some instances of this embodiment, the multi-fluorescence spectra is a fluorescence spectra that is generated by fluorescence emitted from a plurality of photo-activated or photo-excited light-emissive components such as for examples, fluorophores or dyes. The methods, processes and systems of the present technology allow to obtain more accurate information from multi-fluorescence spectra which may be obtained from a fluorescence detection device such as for example, a flow cytometer.

The present technology provides a method to determine the energy transfer occurring between the light-emissive components of the multi-fluorescence spectra. In some instances, the method accounts for stochastic distribution or stochastic concentration of the light-emissive components. The method allows to compensate the multi-fluorescent spectra for stochastic energy transfer.

In some other implementations, the plurality of light-emissive components comprises at least four light-emissive components. At least some of the light-emissive components spectrally overlap. At least some of the light-emissive components have a different light absorption and emission spectrum. Some of the light-emissive components in the plurality of light-emissive components act as energy donor while other light-emissive components act as energy acceptor.

In some implementations, the method accounts for efficiency of energy transfer between pairs of light-emissive components. In some of the same instances, the step of accounting for efficiency of energy transfer between pairs of light-emissive components includes determining ensemble multicolor FRET efficiency. The present technology provides a model to determine the ensemble multi-fluorescence between the light-emissive components of the multi-fluorescence spectra and the imaging device. In some instances, the model accounts for stochastic distribution or stochastic concentration of the light-emissive components. The model allows to compensate the multi-fluorescent spectra for stochastic energy transfer. In some instances, the model is an emFRET model.

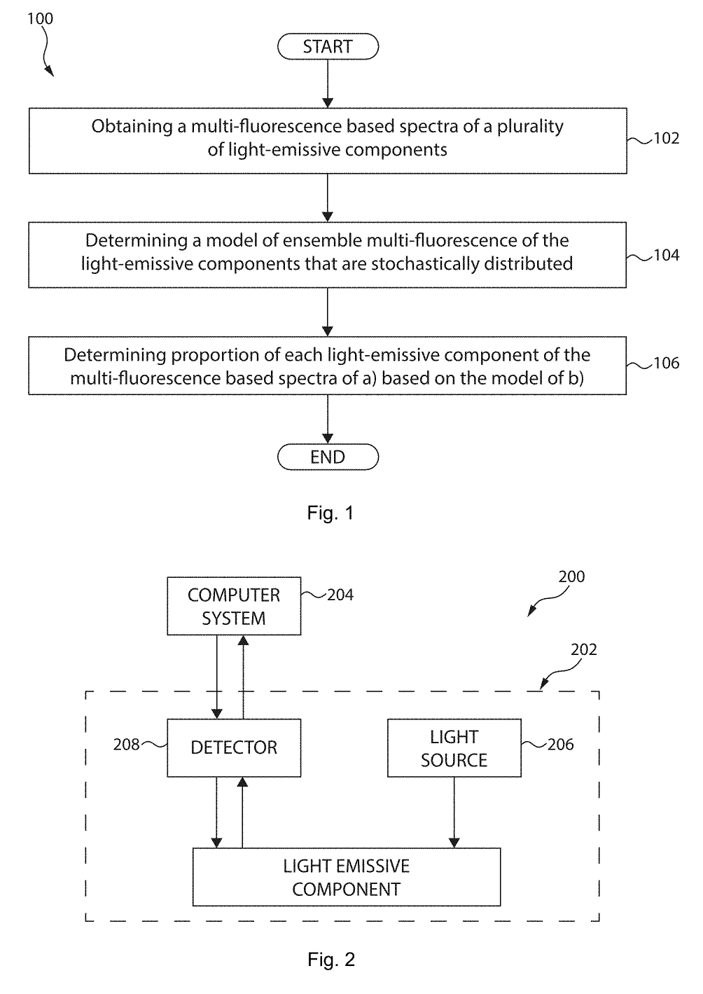

An embodiment of the method provided by the present technology is depicted in FIG. 1, wherein the method 100 comprises a step 102 of obtaining a multi-fluorescence based spectra of a plurality of light-emissive components. The method further comprises a step 104 of determining a model of ensemble multi-fluorescence of the light-emissive components that are stochastically distributed and then a step 106 of determining proportion of each light-emissive component of the multi-fluorescence based spectra of a) based on the model of b).

In some other embodiments, the present technology relates to a method for accurately designing microparticle barcodes as defined by unique ensemble multi-fluorescence spectra through the compensating of stochastic energy transfer by the light-emissive components that have been immobilized on microparticles. The method comprises obtaining a first calibration data for each of the plurality of light-emissive components as imaged by the imaging system. The method then comprises obtaining a second calibration data for pairs of light-emissive components, which models the propensity of energy transfer for the pair in question as imaged by the imaging system. The method then comprises developing a model for stochastic energy transfer based on the first and second calibration data. Thereafter, the ensemble multi-fluorescence of BMPs can be designed in-silico, compensating for energy transfer to yield distinguishable barcodes.

A system for compensating for stochastic energy transfer in multicolor microparticle samples, wherein the light-emissive components have been immobilized on microparticles. The method comprising a processing unit; and a non-transitory computer-readable memory having stored thereon program instructions executable by the processing unit for: obtaining base color data for each of the multicolor microparticle samples, the base color data produced by the application of a plurality of light-emissive components to the multicolor microparticle samples; obtaining first calibration data from a first interaction between the multicolor microparticle samples and a first light source having a first predetermined wavelength; obtain second calibration data from a second interaction between the multicolor microparticle samples and a second light source having a second predetermined wavelength; developing a model for stochastic energy transfer based on the first and second calibration data; and compensating the base color data using the model.

In another embodiment, the present technology provides a method for the detection a multiplicity of surface markers stochastically distributed on a biological substrate such as, for example, cells. Multicolor particles with similar sizes to the cells may be used to calibrate and determine the multi-fluorescence model corresponding to the dyes and the imaging set-up. Thereafter, the cells may be labeled with multiplicity of dye-labeled antibodies (or any affinity binders). Using the model, the multi-fluorescent spectra of every detected cell, and the assumption of stochastic distribution, the concentration of surface markers may be detected.

In another embodiment, the assumption of stochastic distribution may be tested to determine colocalization between two dyes, and corresponding proteins on the surface markers. After calibration of the model, the multi-fluorescent spectra may be tested against the model to determine the presence of co-localization or otherwise interaction between two markers.

The present technology also provides a system for optimizing detection of light-emissive components of a multi-fluorescence spectra, the system comprising a computer system having a processor, the computer system operatively communicable with an imaging device for generating multi-fluorescence based spectra of a plurality of light-emissive components, the processor arranged to execute a method comprising: a) obtaining the multi-fluorescence based spectra of a plurality of light-emissive components; b) determining a model of ensemble multi-fluorescence of the light-emissive components that are stochastically distributed; and c) determining proportion of each light-emissive component of the multi-fluorescence based spectra of a) based on the model of b).

In certain embodiments, the computer system and the imaging system are integral. In certain embodiments, the computer system and the imaging system are physically distinct. The computer system may be a server, or a computer readable medium. The imaging system may comprise a light source for activating the light-emissive components. The imaging system may include a detector for detecting a light emitted by the activated light-emissive components. In certain embodiments, the imaging system is a flow cytometer.

One embodiment of the system of the present technology is depicted in FIG. 2, wherein the system 200 comprises a computer system 204 and an imaging system 202. The computer system 204 comprises a processor. The imaging system 202 comprises a light source 206 for photo-activating the light-emissive components as defined herein and a detector 208 for detecting fluorescence emitted by the photo-activated light-emissive components. The computer system 204 is in operative communication with the imaging system 202 for inter alia generating multi-fluorescence based spectra of a plurality of light-emissive components and detecting the fluorescence emitted by the light-emissive components. The processor is arranged to execute a method comprising: a) obtaining the multi-fluorescence based spectra of a plurality of light-emissive components; b) determining a model of ensemble multi-fluorescence of the light-emissive components that are stochastically distributed; and c) determining proportion of each light-emissive component of the multi-fluorescence based spectra of a) based on the model of b).

In some embodiments, the present technology also provides a kit for calibrating a multi-fluorescence model of a multitude of a multitude of light-emissive components and an imaging device. In some instances, the kit comprises a first set of particles labeled with the individual light-emissive components (i.e., single-color beads) and a second set of particles labeled with at least some pairs of the light-emissive components.

The light-emissive components include components with at least one detectable excitation wavelength and at least one detectable emission wavelength different from the excitation wavelength. In some instances, the light-emissive component is a single molecule. Examples of light-emitting components that may be used in the method of the present technology include fluorescent entities (fluorophores) or phosphorescent entities, for example, cyanine dyes (e.g., FAM, Cy2, Cy3, Cy5, Cy5.5, Cy7, or the like.) metal nanoparticles, semiconductor nanoparticles or “quantum dots,” or fluorescent proteins such as GFP (Green Fluorescent Protein). Other non-limiting examples of potentially suitable light-emissive components include 1,5 IAEDANS, 1,8-ANS, 4-Methylumbelliferone, 5-carboxy-2,7-dichlorofluorescein, 5-Carboxyfluorescein (5-FAM), 5-Carboxynapthofluorescein, 5-Carboxytetramethylrhodamine (5-TAMRA), 5-FAM (5-Carboxyfluorescein), 5-HAT (Hydroxy Tryptamine), 5-Hydroxy Tryptamine (HAT), 5-ROX (carboxy-X-rhodamine), 5-TAMRA (5-Carboxytetramethylrhodamine), 6-Carboxyrhodamine 6G, 6-CR 6G, 6-JOE, 7-Amino-4-methylcoumarin, 7-Aminoactinomycin D (7-AAD), 7-Hydroxy-4-methylcoumarin, 9-Amino-6-chloro-2-methoxyacridine, ABQ, Acid Fuchsin, ACMA (9-Amino-6-chloro-2-methoxyacridine), Acridine Orange, Acridine Red, Acridine Yellow, Acriflavin, Acriflavin Feulgen SITSA, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alizarin Complexon, Alizarin Red, AMC, AMCA-S, AMCA (Aminomethylcoumarin), AMCA-X, Aminoactinomycin D, Aminocoumarin, Aminomethylcoumarin (AMCA), Anilin Blue, Anthrocyl stearate, APTRA-BTC, APTS, Astrazon Brilliant Red 4G, Astrazon Orange R, Astrazon Red 6B, Astrazon Yellow 7 GLL, Atabrine, ATTO 390, ATTO 425, ATTO 465, ATTO 488, ATTO 495, ATTO 520, ATTO 532, ATTO 550, ATTO 565, ATTO 590, ATTO 594, ATTO 610, ATTO 611X, ATTO 620, ATTO 633, ATTO 635, ATTO 647, ATTO 647N, ATTO 655, ATTO 680, ATTO 700, ATTO 725, ATTO 740, ATTO-TAG CBQCA, ATTO-TAG FQ, Auramine, Aurophosphine G, Aurophosphine, BAO 9 (Bisaminophenyloxadiazole), BCECF (high pH), BCECF (low pH), Berberine Sulphate, Bimane, Bisbenzamide, Bisbenzimide (Hoechst), bis-BTC, Blancophor FFG, Blancophor SV, BOBO-1, BOBO-3, Bodipy 492/515, Bodipy 493/503, Bodipy 500/510, Bodipy 505/515, Bodipy 530/550, Bodipy 542/563, Bodipy 558/568, Bodipy 564/570, Bodipy 576/589, Bodipy 581/591, Bodipy 630/650-X, Bodipy 650/665-X, Bodipy 665/676, Bodipy Fl, Bodipy FL ATP, Bodipy Fl-Ceramide, Bodipy R6G, Bodipy TMR, Bodipy TMR-X conjugate, Bodipy TMR-X, SE, Bodipy TR, Bodipy TR ATP, Bodipy TR-X SE, BO-PRO-1, BO-PRO-3, Brilliant Sulphoflavin FF, BTC, BTC-5N, Calcein, Calcein Blue, Calcium Crimson, Calcium Green, Calcium Green-1 Ca2+ Dye, Calcium Green-2 Ca2+, Calcium Green-5N Ca2+, Calcium Green-C18 Ca2+, Calcium Orange, Calcofluor White, Carboxy-X-rhodamine (5-ROX), Cascade Blue, Cascade Yellow, Catecholamine, CCF2 (GeneBlazer), CFDA, Chromomycin A, Chromomycin A, CL-NERF, CMFDA, Coumarin Phalloidin, CPM Methylcoumarin, CTC, CTC Formazan, Cy2, Cy3.18, Cy3.5, Cy3, Cy5.18, cyclic AMP Fluorosensor (FiCRhR), Dabcyl, Dansyl, Dansyl Amine, Dansyl Cadaverine, Dansyl Chloride, Dansyl DHPE, Dansyl fluoride, DAPI, Dapoxyl, Dapoxyl 2, Dapoxyl 3′ DCFDA, DCFH (Dichlorodihydrofluorescein Diacetate), DDAO, DHR (Dihydrorhodamine 123), Di-4-ANEPPS, Di-8-ANEPPS (non-ratio), DiA (4-Di-16-ASP), Dichlorodihydrofluorescein Diacetate (DCFH), DiD-Lipophilic Tracer, DiD (DiIC18(5)), DIDS, Dihydrorhodamine 123 (DHR), DiI (DiIC18(3)), Dinitrophenol, DiO (DiOC18(3)), DiR, DiR (DilC18(7)), DM-NERF (high pH), DNP, Dopamine, DTAF, DY-630-NHS, DY-635-NHS, DyLight 405, DyLight 488, DyLight 549, DyLight 633, DyLight 649, DyLight 680, DyLight 800, ELF 97, Eosin, Erythrosin, Erythrosin ITC, Ethidium Bromide, Ethidium homodimer-1 (EthD-1), Euchrysin, EukoLight, Europium (III) chloride, Fast Blue, FDA, Feulgen (Pararosaniline), FIF (Formaldehyd Induced Fluorescence), FITC, Flazo Orange, Fluo-3, Fluo-4, Fluorescein (FITC), Fluorescein Diacetate, Fluoro-Emerald, Fluoro-Gold (Hydroxystilbamidine), Fluor-Ruby, Fluor X, FM 1-43, FM 4-46, Fura Red (high pH), Fura Red/Fluo-3, Fura-2, Fura-2/BCECF, Genacryl Brilliant Red B, Genacryl Brilliant Yellow 10GF, Genacryl Pink 3G, Genacryl Yellow 5GF, GeneBlazer (CCF2), Gloxalic Acid, Granular blue, Haematoporphyrin, Hoechst 33258, Hoechst 33342, Hoechst 34580, HPTS, Hydroxycoumarin, Hydroxystilbamidine (FluoroGold), Hydroxytryptamine, Indo-1, high calcium, Indo-1, low calcium, Indodicarbocyanine (DiD), Indotricarbocyanine (DiR), Intrawhite Cf; JC-1, JO-JO-1, JO-PRO-1, LaserPro, Laurodan, LDS 751 (DNA), LDS 751 (RNA), Leucophor PAF, Leucophor SF, Leucophor WS, Lissamine Rhodamine, Lissamine Rhodamine B, Calcein/Ethidium homodimer, LOLO-1, LO-PRO-1, Lucifer Yellow, Lyso Tracker Blue, Lyso Tracker Blue-White, Lyso Tracker Green, Lyso Tracker Red, Lyso Tracker Yellow, LysoSensor Blue, LysoSensor Green, LysoSensor Yellow/Blue, Mag Green, Magdala Red (Phloxin B), Mag-Fura Red, Mag-Fura-2, Mag-Fura-5, Mag-Indo-1, Magnesium Green, Magnesium Orange, Malachite Green, Marina Blue, Maxilon Brilliant Flavin 10 GFF, Maxilon Brilliant Flavin 8 GFF, Merocyanin, Methoxycoumarin, Mitotracker Green FM, Mitotracker Orange, Mitotracker Red, Mitramycin, Monobromobimane, Monobromobimane (mBBr-GSH), Monochlorobimane, MPS (Methyl Green Pyronine Stilbene), NBD, NBD Amine, Nile Red, Nitrobenzoxadidole, Noradrenaline, Nuclear Fast Red, Nuclear Yellow, Nylosan Brilliant lavin E8G, Oregon Green, Oregon Green 488-X, Oregon Green, Oregon Green 488, Oregon Green 500, Oregon Green 514, Pacific Blue, Pararosaniline (Feulgen), PBFI, Phloxin B (Magdala Red), Phorwite AR, Phorwite BKL, Phorwite Rev, Phorwite RPA, Phosphine 3R, PKH26 (Sigma), PKH67, PMIA, Pontochrome Blue Black, POPO-1, POPO-3, PO-PRO-1, PO-PRO-3, Primuline, Procion Yellow, Propidium lodid (PI), PyMPO, Pyrene, Pyronine, Pyronine B, Pyrozal Brilliant Flavin 7GF, QSY 7, Quinacrine Mustard, Resorufin, RH 414, Rhod-2, Rhodamine, Rhodamine 110, Rhodamine 123, Rhodamine 5 GLD, Rhodamine 6G, Rhodamine B, Rhodamine B 200, Rhodamine B extra, Rhodamine BB, Rhodamine BG, Rhodamine Green, Rhodamine Phallicidine, Rhodamine Phalloidine, Rhodamine Red, Rhodamine WT, Rose Bengal, S65A, S65C, S65L, S65T, SBFI, Serotonin, Sevron Brilliant Red 2B, Sevron Brilliant Red 4G, Sevron Brilliant Red B, Sevron Orange, Sevron Yellow L, SITS, SITS (Primuline), SITS (Stilbene Isothiosulphonic Acid), SNAFL calcein, SNAFL-1, SNAFL-2, SNARF calcein, SNARFI, Sodium Green, SpectrumAqua, SpectrumGreen, SpectrumOrange, Spectrum Red, SPQ (6-methoxy-N-(3-sulfopropyl)quinolinium), Stilbene, Sulphorhodamine B can C, Sulphorhodamine Extra, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, Tetracycline, Tetramethylrhodamine (TAMRA), Texas Red, Texas Red-X conjugate, Thiadicarbocyanine (DiSC3), Thiazine Red R, Thiazole Orange, Thioflavin 5, Thioflavin S, Thioflavin TCN, Thiolyte, Thiozole Orange, Tinopol CBS (Calcofluor White), TMR, TO-PRO-1, TO-PRO-3, TO-PRO-5, TOTO-1, TOTO-3, TRITC (tetramethylrodamine isothiocyanate), True Blue, TruRed, Ultralite, Uranine B, Uvitex SFC, WW 781, X-Rhodamine, XRITC, Xylene Orange, Y66F, Y66H, Y66 W, YO-PRO-1, YO-PRO-3, YOYO-1, YOYO-3, SYBR Green, Thiazole orange (interchelating dyes), or combinations thereof.

The emFRET model results in an accessible analytical solution and provides quantitative insight into stochastic mFRET cascades, allowing rational design and fine-tuning of the spectral response. The barcoding platform described herein enables effective use of common, spectrally overlapping dyes by extending barcoding into extreme FRET regimes, and provides a direct path for expanding the barcoding capacity.

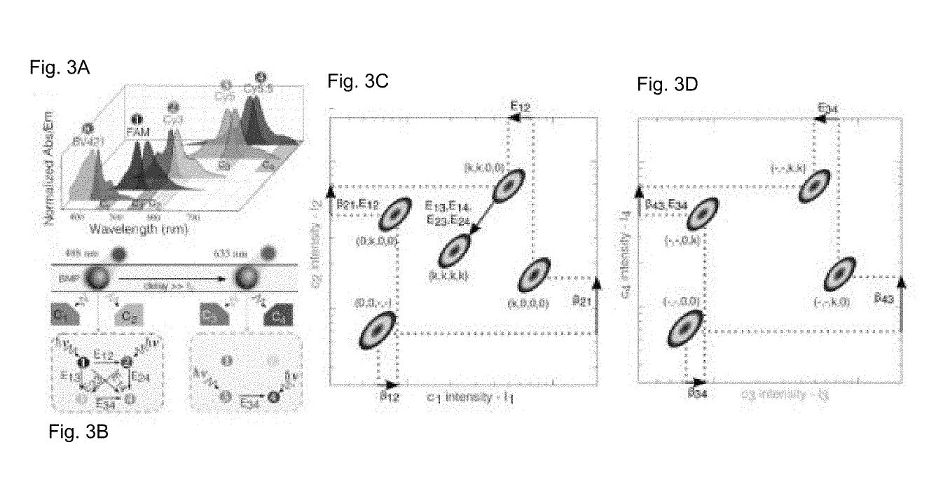

To best illustrate the problem of mFRET in barcoding, a high capacity barcoding system was designed with spectrally overlapping dyes. In order of increasing wavelength, the four chosen classifier dyes are FAM, Cy3, Cy5, and Cy5.5, referred from here on as dyes 1 to 4 respectively (FIG. 3A and Table 1).

TABLE 1
Fluorophore photophysical properties and interrogation/read-out.

| Fluorophore | Ext. coeff. | Abs max | Em max | Laser | Filter | R_{da} (in Å) | | | |
|---|---|---|---|---|---|---|---|---|---|
| (donor) | (M⁻¹cm⁻¹) | λₘₐₓ (nm) | λₘₐₓ (nm) | (nm) | (nm) | FAM | Cy3 | Cy5 | Cy5.5 |
| FAM | 75,000 | 492 | 518 | 488 | 530/30 | — | 55 | 45 | 43 |
| Cy3 | 150,000 | 552 | 568 | 488 | 585/42 | — | — | 53 | 49 |
| Cy5 | 250,000 | 652 | 671 | 633 | 660/20 | — | — | — | 67 |
| Cy5.5 | 209,000 | 678 | 696 | 633 | 780/60 | — | — | — | — |

This classifier dye configuration allows excitation and readout using common lasers and optical filters respectively, achieving pairwise excitation of (1,2) and (3,4) using blue (488 nm) and red (633 nm) lasers, respectively, and pairwise readout using channels c1-c2 and c3-c4, respectively (FIG. 1B). BV-421 was used as assay reporter dye as it is bright and excited at 405 nm. The dyes' absorption/emission spectra showcase the substantial spectral overlap, and the calculated Förster radii R_{da} (where d and a are the donor and acceptor respectively) underline their propensity for energy transfer (Table 1). Spectral barcodes are generated by modulating the proportions of the dyes surface density, (δ₁, δ₂, δ₃, δ₄), to generate well-resolved clusters arising from the intensity scatter plots in each channel pair, thereby allowing unambiguous decoding of the BMPs. Bleed-through and FRET, however, break down the orthogonality between the density (δ_{f}) and the cognate channel intensity (I_{f}), for a given dye f, which prevents straightforward barcoding (FIGS. 3C and 3D). Bleed-through is a linear effect at the detector level and can readily be accounted for by solving simultaneous linear equations. FRET, on the other hand, breeds a non-linear response to the dyes' surface densities and cannot be deconvolved as simply (FIGS. 3C-3D).

In this example, laser excitation at 488 nm results in 6 potential inter-dye energy transfers with varying efficiencies, E_{da}, between donor d and acceptor a (FIG. 3B). Hence, barcode responses in the I₁-I₂ intensity space are also dependent on (δ₃, δ₄) through the density-dependent energy transfer pathways E₁₃, E₁₄, E₂₃ and E₂₄ (FIG. 3C). Without an accurate model to guide the design process, the non-linear nature of FRET imposes empirical optimizations of (δ₁, δ₂) values for every (δ₃, δ₃) value. Furthermore, the number of optimization steps increases exponentially with every added classifier dye, and collectively justify the current practice that focuses on minimizing spectral overlap and mFRET, albeit at the expense of barcoding capacity.

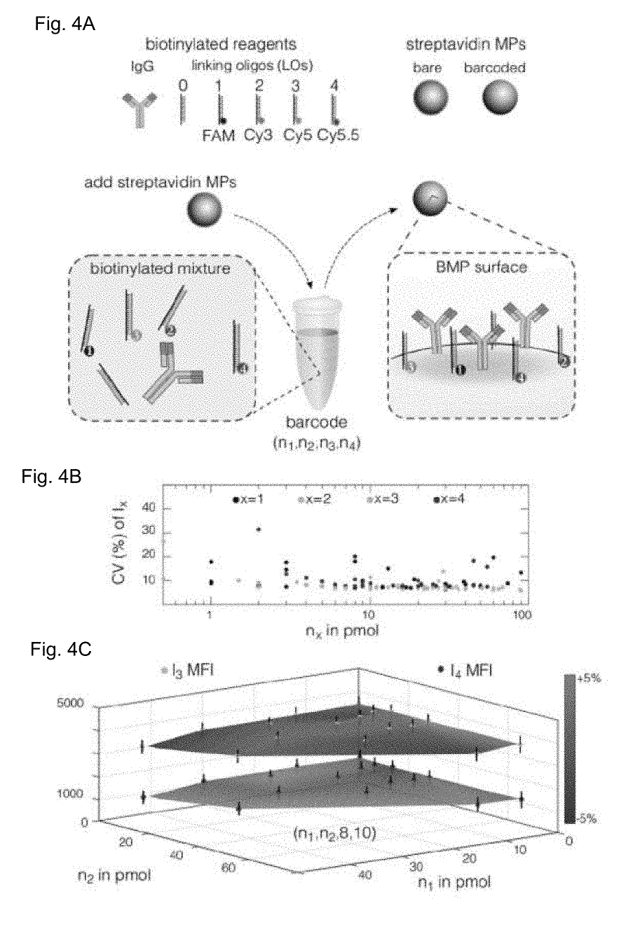

To establish a mechanistic mFRET model for surface immobilized dyes, it is necessary to achieve accurate control over dye proportions, which is a requisite not met by commonly used labelling techniques. A widely applicable one-pot microparticle labelling method was designed. All classifier dyes were linked to the 3′ end of an identical 21-nt DNA oligonucleotide that, when annealed to its complimentary 5′ biotinylated strand, served as a linker oligonucleotide (LO) for streptavidin coupled MPs. DNA is used solely as a homogeneous crosslinker to normalize reactivity and footprint across all classifier dyes. Each classifier dye (1-4) was thus respectively conjugated to a LO (LO₁-LO₄). A non-fluorescently-labeled LO (LO₀) was also used to balance and conserve the total amount of LOs in solution, which consequently conserves the total LO density on the MPs, independent of the particular barcode (FIG. 4A). As a result, the proportions of colored LOs in solution, given by (η₁, η₂, η₃, η₄) and defining the barcode, are translated to the MP after labelling, that is, (δ₁, δ₂, δ₃, δ₄)=t×(η₁, η₂, η₃, η₄), where t is a labelling constant. BMPs co-labeled with mouse anti-goat IgG and LOs were characterized by cytometry. The coefficient-of-variation of bead-to-bead intensity in each respective detector, which defines the cluster size, was ≈10% and barcode-independent (FIG. 4B).

To verify that the surface densities of differently-colored LOs were independent, I₃-I₄ for a set of BMPs with constant (η₃, η₄) but varying (η₁, η₂) were measured. The response of BMPs in c₃ and c₄ is neither impacted by bleed-through nor FRET from dyes 1 and 2 due to the spatial and temporal separation of the two excitation cells (FIG. 3B). Hence, any detectable dependence of I₃-I₄ on (η₁, η₂) can be unambiguously be ascribed to changes in (δ₃, δ₄) because of surface competition. FIG. 4C, shows that the intensity of I₃-I₄ was constant for a wide range of η₁ and η₂ in barcodes (η₁, η₂, 8, 10), thus demonstrating independence of the dyes surface densities and conservation of dye proportions during the labelling reaction. In addition, the conservation of total LOs also ensured barcode-independent antibody surface coverage, which was measured by targeting the IgGs with Goat anti-Mouse (GAM) secondary-Ab labeled with BV-421.

To model ensemble multi-fluorescence spectra, a general, platform-independent multicolor fluorescence model (MFM) was derived that links the fluorescence intensities of every channel to the barcode-specific relative dye densities. The MFM considers direct (i.e. laser) excitation of dyes as well as sensitization by FRET, mFRET cascades, and the platform specific bleed-through parameters. Here, the signal in a given channel is assumed to be registered in response to only one laser. A MFM with a higher degree of generality, also considering channel intensities in response to an arbitrary number of lasers, is derived. Briefly, the signal in a given channel is modeled as the sum of the ensemble fluorescence intensities of N distinct dyes. Accordingly, the equation for the intensity of channel c can be expressed as:

$${I}_{c} = {I}_{c}^{0} + {\sum }_{f = 1}^{N} {β}_{cf} {F}_{f}^{e} , c = 1 , … C , f = 1 , … , N \qquad ( 1 )$$

where I_{c} and I_{c}^{o} are, respectively, the signal and background (i.e. bare MPs) in channel c when excited by the channel-specific laser, F^{e}_{f} is the ensemble fluorescence of dye f, and β_{cf} is the bleed-through ratio in channel c from dye f.

To account for FRET cascades, the sensitized fluorescence, F^{s}_{f}, denotes the unattenuated ensemble emission (that is, considering only radiative decay) of dye f and is modeled as the sum of direct excitation as well as FRET excitation from all potential acceptors, which is a simplification afforded by the low exciton density:

$$\begin{matrix}{F}_{f}^{e} = {F}_{f}^{s} ( 1 - {\sum }_{i = f + 1}^{N} {E}_{fi} ) & ( 2 a ) \\ {F}_{f}^{s} = {F}_{f}^{0} + {\sum }_{j = 1}^{f - 1} {α}_{jf} {E}_{jf} {F}_{j}^{s} & ( 2 b )\end{matrix}$$

where E^{em}_{da} is the ensemble-average of the FRET efficiency in its classic form (namely, that a de-excitation of the donor d will directly result in the excitation of acceptor a), α_{da} is the FRET proportionality constant that depends on the dyes mutual optical properties and which can be seen as an ‘energy exchange rate’, and F⁰_{f} is the basal fluorescence due to direct excitation. These equations model steady-state FRET cascades whereby excitons may undergo multiple transfers before radiative emission.

It was considered operation in the linear regime whereby the basal fluorescence of dye f will be proportional to its surface density (i.e. F⁰_{f} α σ_{f}). By considering that σ_{f}=tn_{f}, which is afforded by the labelling reaction as discussed hereinabove, the basal fluorescence may be expressed as F⁰_{f}=μ_{f}n_{f}, where μ_{f} is a dye- and laser dependent direct-excitation constant.

To use the MFM, it is necessary to establish the energy transfer distribution in FIG. 4A and FIG. 4B as a function of the dyes surface densities. Ensemble 2FRET (e2FRET), whereby donor molecules may transfer excitons to an array of stochastically distributed acceptors with a constant Förster radius, are mechanistically described using an ensemble average of single-donor environments in 3D and 2D. The resulting e2FRET efficiency (E^{e}_{da}) can be calculated using a power series expansion or a practical closed-form approximation. Importantly, E^{e}_{da} is only dependent on the average number of acceptor molecules in an R_{da} radius around the donor, ω_{da}, which is a dimensionless number that naturally emerges from the model. Hence, ω_{da}=πσₐR²_{da} and is referred from hereon as the “Förster acceptor number” given its relation to R_{da}.

As disclosed herein, the model is extended and the ensemble multicolor FRET (emFRET) efficiency between N differently-colored dyes that are stochastically distributed on a planar surface (2D) was derived. Notably, the impact of multicolor acceptors on the total FRET efficiency (E^{em}_{d}) for a given donor is found to be a simple addition of the pairwise Förster acceptor numbers, yielding ω^{m}_{d}=Σₐ ω_{da} where ω^{m}_{d} is the total Förster acceptor number. This finding is directly equivalent to an e2FRET scenario with a single effective acceptor species and an effective Förster radius (R^{e}), as depicted in FIG. 3A. Unlike the classical definition of Förster radius, R^{e}_{d} is dependent on the surface density of acceptors as well as the spectral overlap of the dyes involved. The ensemble average calculation is predicated on satisfying the conditions of (i) non-saturated exciton density, (ii) random dye distributions, (iii) independent surface densities for the differently, colored dyes, and (iv) isotropic dye orientations, which are all met by the labelling method. The total emFRET efficiency for a donor d to all potential acceptors can then be readily calculated using the closed-form e2FRET expression after substitution with the donor-specific ω^{m}_{d},

$${E}_{d}^{T} = \frac{{( {ω}_{d}^{T} / γ )}^{λ}}{1 + {( {ω}_{d}^{T} / γ )}^{λ}} \qquad ( 3 )$$

where γ and λ are the exclusion and fitting constants, respectively. Using equation (3), it was determined that the total energy is transferred to the different acceptor species proportionally to (ω_{da}/ω^{d}^{_{f}})^{λ} allowing quick determination of energy transfer distributions and cascades from the surface densities, and imparting mechanistic insight to complex multicolor interactions.

The emFRET model constitutes the ‘kernel’ of the MFM, which can be calculated after determining the values of the photophysical parameters (i.e. cytometer and classifier dyes) in a one-time calibration experiment. As many parameters take a zero value in a setup such as the flow cytometer used here, the algebraic equations constituting the MFM are greatly simplified. All non-zero parameters were determined using 18 judiciously selected barcodes.

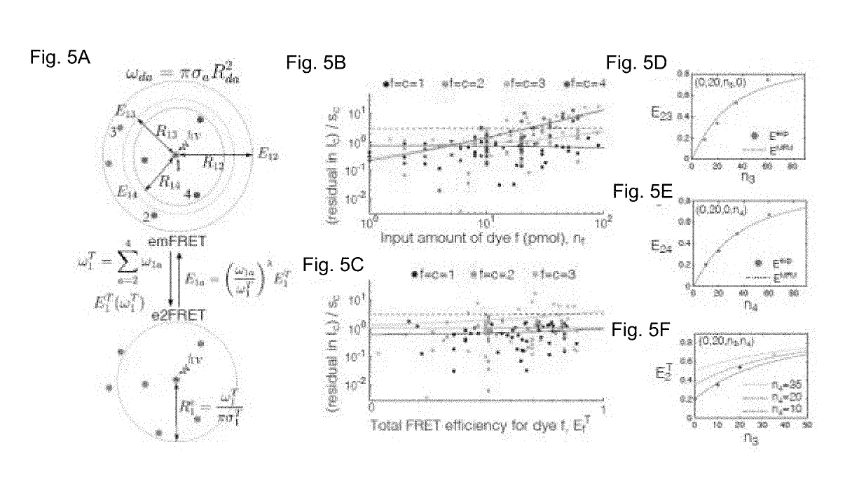

The accuracy of the MFM was evaluated by comparing the predicted and measured fluorescence for a number of arbitrary four-color BMPs and calculating, for every channel c, the residual error normalized by the standard deviation (s_{c}) of the bead intensities. The residual error was typically <3×s_{c} for most conditions, which is adequate for barcoding applications. The general trend of the error in channel c was plotted against n_{f} for f=c (FIG. 5B) showing that the residual errors of I₁ and I₂ are independent of n₁ and n₂, whereas the residual error in I₃ and I₄ increases beyond 3×s_{c} at high concentrations of dyes 3 and 4 respectively, which points to a breakdown in the linear basal fluorescence approximation and which it was ascribe to self-quenching in these dyes. As plotted in FIG. 5C, an increase in the calculated total FRET efficiency for the donor dyes (i.e. dyes 1-3) did not lead to an increase in the normalized residual error, indicating that the accuracy of the emFRET model is steady from low to extreme FRET levels. Next, the accuracy of the MFM-computed FRET efficiency (E^{MFM}) in barcodes was tested such that it permits FRET efficiency calculation using a donor quenching method. Experimental FRET efficiencies (E^{exp}), averaged over bead ensembles, were found to be in good agreement with the emFRET model, as shown in FIGS. 5D-5F.

Following calibration and validation of the MFM, the spectral positions of barcode clusters were predicted simply from their starting dye amounts, enabling barcode design with high accuracy to maximize the barcoding capacity. The barcodes were iteratively optimized in silico, which in effect permits anticipation and compensation for emFRET effects, and thus enables barcoding at regimes with very high mFRET.

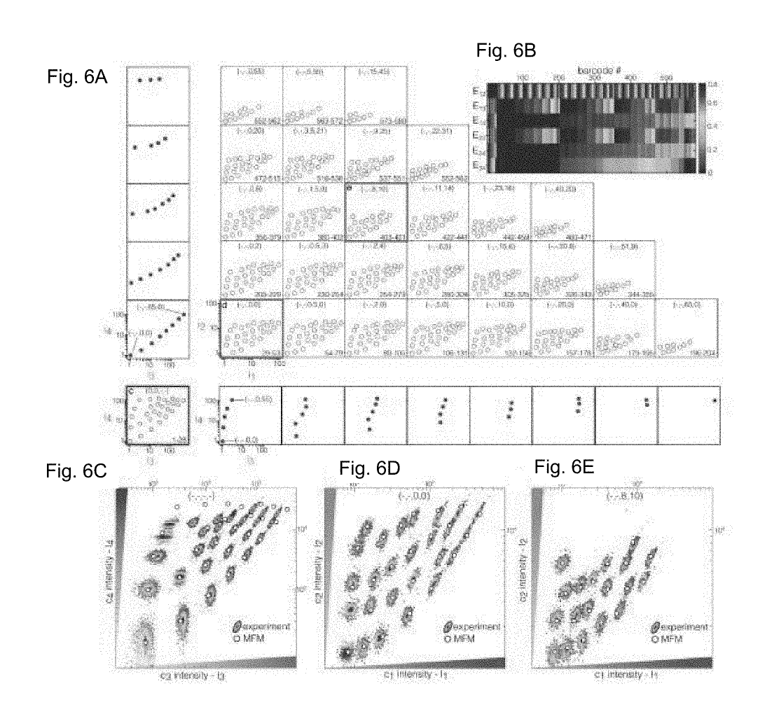

Following in silico optimization, 580 barcodes with well-resolved regions were generated (FIG. 6A). The six inter-dye emFRET efficiencies predicted within each barcode highlight the strong energy transfer which reaches 76% at its maximum (FIG. 4B). As expected, the maximal levels of emFRET reached between two dyes is commensurate to their calculated single-molecule Förster radius and density. The predicted barcode intensities are superimposed over the measured scattered intensity plots of their associated BMPs, showing a very good overall agreement (FIGS. 6C-6E). The clusters in I₃-I₄ deviated from their predicted values at high concentrations (see FIG. 5B) due to the self-quenching and breakdown of the linear basal fluorescence response. The clusters in I₁-I₂ were in very good agreement with the predicted regions as seen in FIG. 6C and FIG. 6D for barcodes (n₁, n₂, 0, 0) and (n₁, n₂, 8, 10) respectively. These results demonstrate the power of the emFRET model for rapid, high capacity barcoding with spectrally overlapping dyes with emFRET levels of up to at least 76%. Absent a model, barcoding at high density becomes problematic when the total FRET efficiency for a given dye exceeds the inherent variability of barcode responses (i.e. when E^{T}≥CV˜10%). Of the designed 580 barcodes, only 67 barcodes incur less than 10% of total FRET efficiency for any given dye (i.e. E^{T}<10%), which suggests a ˜9× increase in capacity thanks to the MFM.

To benefit from the throughput of flow cytometry and high capacity barcodes, automated decoding is imperative, but has not been possible to date for barcodes subject to mFRET. Automated decoding entails (i) clustering the BMP dataset, (ii) classifying the BMPs into the different clusters, and (iii) assigning these clusters- and thus the BMPs within-to their cognate barcodes. Whereas (i) and (ii) are straightforward with orthogonal classifier dyes, these tasks develop into a multivariate problem in the case of non-orthogonal classifiers, and rapidly become challenging and computationally expensive. Furthermore, (iii) is impossible without a priori knowledge of the relative barcode responses; as a result, the hitherto intractable ensemble fluorescence caused by mFRET have required cluster assignment to be manually initialized for every experiment, even for relatively low FRET levels. We sought to leverage the MFM to fully automate the decoding of BMPs.

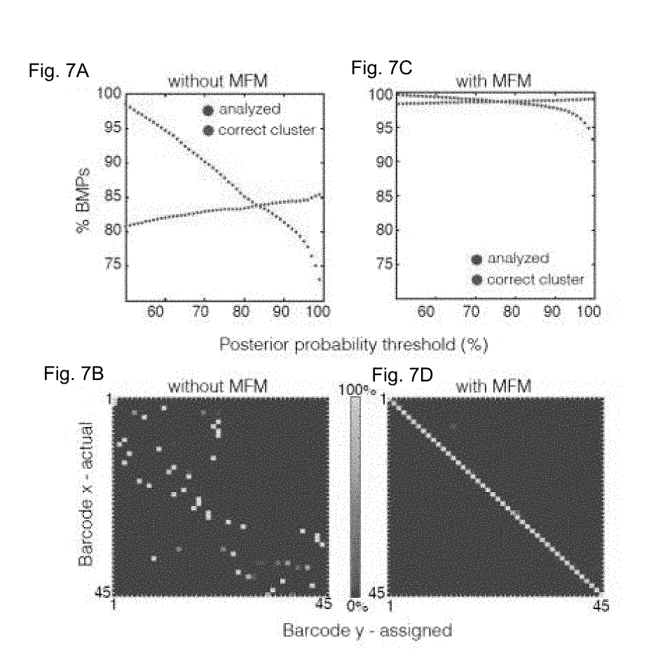

To decode BMPs based on 4D intensity data (I₁, I₂, I₃, I₄), a sequential 2D clustering was performed, classification and assignment of BMPs in each of the pairwise channel intensities. Clustering and classification were automated using a Gaussian mixture model (GMM)-based algorithm, whereby BMPs were classified to the clusters in accordance with the highest posterior probability, provided it was higher than the threshold. A digitally-concatenated representative dataset of 45 barcodes was classified in 2D by its I₁-I₂ intensity scatter values, and the fraction of BMPs classified to the correct cluster was quantified. Without the MFM, and thus without prior knowledge of the relative barcode intensities, clustering was inadequate and resulted in significant misclassification (FIG. 7A), as expected. Using the MFM, the predicted barcode intensities can be used as the initial GMM mean value, which led to a deterministic convergence to clusters that yields >99% confidence in BMP classification with minimal BMP exclusions (<5%) (FIG. 7B).

Finally, complete 4D decoding was performed for the same 45 barcode dataset to evaluate the impact of the MFM on automating the assignment step. Without the MFM, and thus without a priori knowledge of the relative intensities, the means of the 2D GMM-clusters were sorted according to their mean values and assigned to the target barcodes. Due to the strongly non-orthogonal response, >90% of BMPs were consistently wrongly decoded (FIG. 7C). On the other hand, the GMM clustering following MFM predictions converged rapidly to their cognate barcodes, thereby simultaneously achieving cluster-to-barcode assignment (FIG. 5D). Importantly, these findings demonstrate completely automated decoding of four-color BMPs under non-orthogonal conditions and strong inter-dye FRET.

Accordingly emFRET model and a microparticle labelling method is provided that together yield a predictive multicolor fluorescence model and enable in silico design, synthesis, and completely automated decoding of fluorescent barcodes. It is shown that by extending barcoding to regimes with extreme FRET efficiencies, the barcoding capacity can be significantly increased. Moreover, it is demonstrated that common dyes with wide spectral response, which historically have been deemed unsuitable for barcoding, may be employed for large scale multiplexing to make use of their wide availability, low cost, and compatibility with flow cytometers. Despite the energy lost to FRET, a ˜20-fold expansion of the barcoding capacity by comparing two-color BMPs (28 FAM/Cy3 barcodes, FIG. 6B) with the four-color BMPs (580 barcodes, FIG. 6A). Furthermore, the platform described herein provides direct means for further addition of dyes; for example, by using near-infrared dyes such as Cy7 and Cy7.5 to generate six-color barcodes. Hence, by extension, it is expected that six-color BMPs would expand the capacity by at least one order of magnitude.

The one-pot synthesis of BMPs using the LOs afforded accurate and independent control of dye densities which was essential to allow mathematical modeling of the BMPs' fluorescence. The LO-based synthesis is easy to implement, employs common organic dyes, yields quick, precise and reproducible results, making it accessible to a wide range of scientists for in-house, large scale multiplexing, barcoding and other applications. The calibration procedure, which is only required once for a specific cytometer and dye configuration, may be performed in under 3 hours. Furthermore, unless the optics are significantly modified, the barcoding capacity should remain unaffected.

It is thus provided a mechanistic model for energy transfer between a multiplicity of dyes composed of an arbitrary number of species that are stochastically distributed in 2D. Using an effective acceptor transform, the emFRET scenario may rewritten as e2FRET by computing the effective Förster radius for every donor species. The emFRET model outlined here imparts insight into multiplexed FRET interactions, and aids in meeting the growing interest to perform multiplex FRET experiments with increasing complexity. The ability to rationally design ensemble mFRET interactions is useful to optimize exciton transfer in dye-sensitized solar cells.

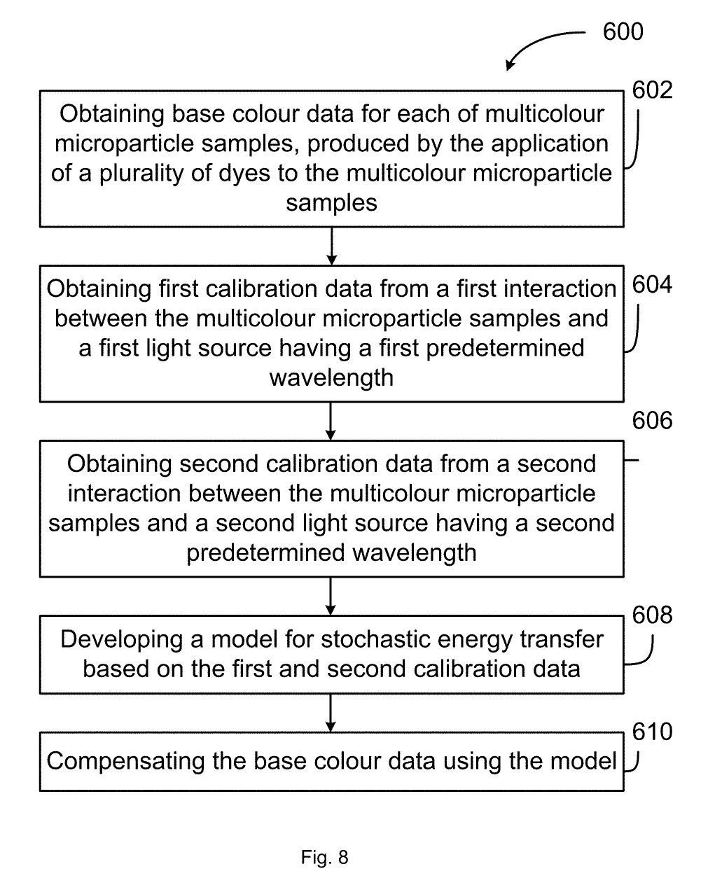

With reference to FIG. 8, there is provided a method 600 for compensating for stochastic energy transfer in multicolor microparticle samples (MMSs). At step 602, base color data for each of the MMSs is obtained. The base color data is produced by the application of a plurality of dyes to the MMS. In some embodiments, each of the MMSs is provided with a different mixture of the plurality of dyes.

At step 604, first calibration data is obtained from a first interaction between the MMSs and a first light source having a first predetermined wavelength. At step 606, second calibration data is obtained from a second interaction between the MMSs and a second light source having a second predetermined wavelength. In some embodiments, the first light source has a wavelength of approximately 488 nm, and the second light source has a wavelength of approximately 633 nm. In some embodiments, the first and second calibration data are multichannel data, that is each of the first and second calibration data is composed of a plurality of sets of data. For instance, the first calibration data is representative of the response of a first subset of the plurality of dyes to the first light source, and the second calibration data is representative of the response of a second subset of the plurality of dyes to the second light source. In some embodiments, the second subset of dyes includes one or more dyes which form the first subset of dyes.

At step 608, a model for stochastic energy transfer is developed based on the first and second calibration data. The model may be the emFRET model as a standalone model or as part of the MFM model. The stochastic energy transfer model can be developed using the approaches outlined in the preceding paragraphs. At step 610, the base color data is compensated using the stochastic energy transfer model developed at step 608.

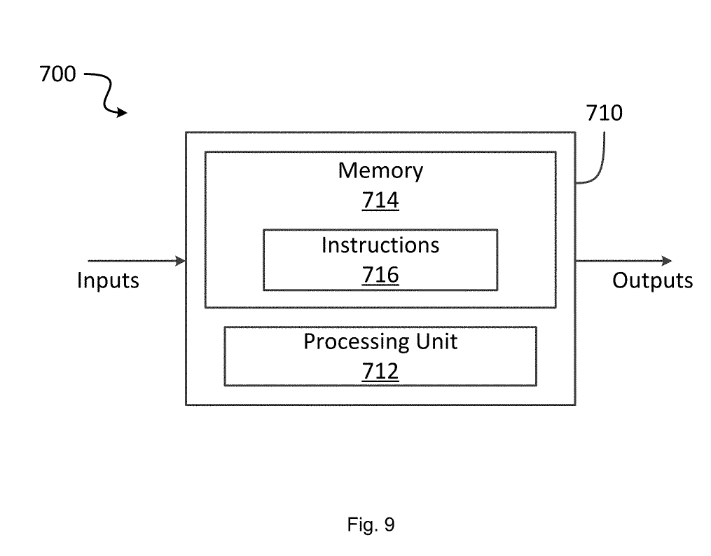

With reference to FIG. 9, the method 600 may be implemented by a computing device 710, comprising a processing unit 712 and a memory 714 which has stored therein computer-executable instructions 716. The processing unit 712 may comprise any suitable devices configured to implement the method 600 such that instructions 716, when executed by the computing device 710 or other programmable apparatus, may cause the functions/acts/steps of the method 600 described herein to be executed. The processing unit 712 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory 714 may comprise any suitable known or other machine-readable storage medium. The memory 714 may comprise non-transitory computer readable storage medium, for example, but not limited to, an electronic, magnetic, optical, electromagnetic, infrared, or semiconductor system, apparatus, or device, or any suitable combination of the foregoing. The memory 714 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 714 may comprise any storage means (e.g., devices) suitable for retrievably storing machine-readable instructions 716 executable by processing unit 712.

The methods and systems for compensating for stochastic energy transfer in multicolor microparticle samples described herein may be implemented in a high level procedural or object oriented programming or scripting language, or a combination thereof, to communicate with or assist in the operation of a computer system, for example the computing device 710. Alternatively, the methods and systems described herein may be implemented in assembly or machine language. The language may be a compiled or interpreted language. Program code for implementing the methods and systems described herein may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems described herein may also be considered to be implemented by way of a non-transitory computer-readable storage medium having a computer program stored thereon. The computer program may comprise computer-readable instructions which cause a computer, or more specifically the processing unit 712 of the computing device 710, to operate in a specific and predefined manner to perform the functions described herein.

Computer-executable instructions may be in many forms, including program modules, executed by one or more computers or other devices. Generally, program modules include routines, programs, objects, components, data structures, etc., that perform particular tasks or implement particular abstract data types. Typically the functionality of the program modules may be combined or distributed as desired in various embodiments.

The above description is meant to be exemplary only, and one skilled in the relevant arts will recognize that changes may be made to the embodiments described without departing from the scope of the invention disclosed. For example, the blocks and/or operations in the flowcharts and drawings described herein are for purposes of example only. There may be many variations to these blocks and/or operations without departing from the teachings of the present disclosure. For instance, the blocks may be performed in a differing order, or blocks may be added, deleted, or modified. While illustrated in the block diagrams as groups of discrete components communicating with each other via distinct data signal connections, it will be understood by those skilled in the art that the present embodiments are provided by a combination of hardware and software components, with some components being implemented by a given function or operation of a hardware or software system, and many of the data paths illustrated being implemented by data communication within a computer application or operating system. The structure illustrated is thus provided for efficiency of teaching the present embodiment. The present disclosure may be embodied in other specific forms without departing from the subject matter of the claims. Also, one skilled in the relevant arts will appreciate that while the systems, methods and computer readable mediums disclosed and shown herein may comprise a specific number of elements/components, the systems, methods and computer readable mediums may be modified to include additional or fewer of such elements/components. The present disclosure is also intended to cover and embrace all suitable changes in technology.

The present description will be more readily understood by referring to the following examples.

### EXAMPLES

### Example 1—Determination of Classifier Dyes

Dyes that emit at closely spaced wavelengths were used so as to help expanding the number of dyes used and, with it, the barcoding capacity. mFRET expected as a consequence of the closely spaced wavelengths chosen served to validate the emFRET model. It was noted that using an assay reporter dye in the blue-shifted spectrum avoids interference with barcodes through analyte-dependent FRET as it cannot act as acceptor to any of the classifier dyes.

### Example 2—Design and Preparation of Linker Oligonucleotides

Linker oligonucleotides (LO_{f}) were formed through hybridization of complementary 21-nt oligos: a 5′ biotinylated oligo (BO) and a fluorescent oligo (FO) 3′-labeled with dye. FO₀ was unlabeled, whereas FO₁—FO₄ where labeled with dyes 1-4 where dye 1 is FAM; dye 2 is Cy3, 3; dye 3 is Cy5; and 4 is Cy5.5 respectively. The BO sequence used was: 5′Biotin/TTTTTTTTTGTGGCGGCGGTG/3′. The fluorescent oligonucleotide sequence was: 5′/CACCGCCGCCACAAAAAAAAA/-f. BOs and FOs were annealed at 10 μM in phosphate buffer saline (PBS)+350 mM NaCl. All oligonucleotides were acquired already modified from Integrated DNA Technologies (IDT, Coralville, Iowa, USA). The sequences were optimized using the mfold web server for minimal secondary structure formation.

### Example 3—Co-Immobilization of Oligonucleotides and Antibodies

A volume of 25 μL of any given barcode (n1, n2, n3, n4), were prepared by mixing biotinylated reagents containing 6.7 pmol of IgG (1 μg), the corresponding amounts of LO_{f}, such that n0+n1+n2+n3+n4=90 pmol and PBS+300 mM NaCl. Next, 25 μL PBS+300 mM NaCl containing 3.25×10⁶ streptavidin-coated superparamagnetic microparticles (M270-Streptavidin from Life Technologies, Carlsbad, Calif., USA) were added, and the reaction tube incubated with end-over-end rotation for one hour, followed by 3 cycles of magnetic aggregation and washing in PBS+0.1 v/v % Tween-20 (PBST). Following synthesis, batches of BMPs were stored separately in the dark at 4° C. and were mixed prior to use in a multiplexed assay. Secondary antibodies were purchased from Life Technologies, whereas all matched antibody pairs and recombinant proteins for multiplexed assays were purchased from Abcam (Cambridge, Mass., USA).

### Example 4—Flow Cytometry

BMPs were read out using the FACS CANTO II cytometer by BD with blue (488 nm) and red (633 nm) lasers. In the blue-laser flow cell, 530/30 and 585/42 band-pass (BP) filters were used for channels 1 and 2, respectively. In the red-laser flow cell, 660/20 and 780/60 were used for channels 3 and 4, respectively. For reporter dye detection during assays, the violet laser (405 nm) was used with a 450/40 BP filter. During validation of the decoding step, BMPs were measured separately and concatenated digitally before any subsequent data analysis.

### Example 5—Single-Molecule Förster Radii

Emission and absorption spectra were used to calculate the overlap integral, J_{da}(λ), and subsequently the single molecule Förster radius R_{da}. The latter was calculated for each donor acceptor pair using the following expression:

R_{da}=9.78×10³(κ²ň⁻⁴Q_{d}J_{da}(λ))^{1/6} (4)

where, κ² is the dipole-dipole orientation factor taken to be as ⅔ as per the dynamic isotropic approximation, ň is the medium refractive index, and Q_{d} is the fluorescence quantum yield of the donors. Absorption and emission spectra of LOs were measured using on a SpectraMax i3x Multi-mode microtiter plate.

### Example 6—Establishment of Ensemble mFRET Model

The total FRET efficiency, E^{T}, from a donor to multicolor acceptors stochastically distributed on a 2D surface was calculated using the probabilistic decay function ρ_{d}(t), which denotes the probability that a donor excited at time t=0 is still excited at t,

$${E}_{d}^{T} = 1 - \frac{1}{{τ}_{d}^{0}} {\int }_{0}^{∞} {ρ}_{d} ( t ) dt . \qquad ( 5 )$$

where τ^{o}_{d} is the unperturbed donor lifetime. For an excited donor molecule, the decay function is governed, as per Förster's theory, by the following differential equation:

$$- \frac{d}{dt} {ρ}_{d} ( t ) = ( 1 + {\sum }_{a = d + 1}^{N} {\sum }_{z = 1}^{{Z}_{a}} {( \frac{{R}_{da}}{{r}_{z , a}} )}^{6} ) \frac{{ρ}_{d} ( t )}{{τ}_{d}^{0}} \qquad ( 6 )$$

where Zₐ is the number of acceptors from dye species a in the vicinity of an excited donor d and r_{za} is the distance between donor d and the z-th acceptor of species a. The solution of equation (6) is then ensemble averaged for all donors (i.e. for all potential configurations of acceptors) mirroring the e2FRET derivation by Wolber and Hudson (Wolber & Hudson, 1979, Biophysical Journal, 28: 197-210). The decay function of the donor in an emFRET scenario is equivalent to that of an e2FRET using the transformation on the Förster acceptor number:

$${ω}_{d} \to {ω}_{d}^{T} = {\sum }_{a = d + 1}^{N} {ω}_{d a} \text{?}$$

$$\text{?} \text{indicates text missing or illegible when filed}$$

where ω^{T}_{d}
may be directly plugged in equation (3). Within this transformation, the e2FRET acceptor corresponds to an effective acceptor with an effective Förster radius.

$${R}_{d}^{e} = {( {\sum }_{a = d + 1}^{N} \frac{{R}_{da}^{2} {σ}_{a}}{{σ}_{d}^{T}} )}^{\frac{1}{2}} . \qquad ( 7 )$$

Overall, this derivation shows that the multicolor Förster acceptor number (omega) is equal to the sum of the individual acceptor numbers.

### Example 7—Model Parameterization

Because of the spatially and temporally separate excitation in a flow cytometer and the spectral properties of the dyes in question, a number of variables in the bleed-through and FRET proportionality matrices (B and A respectively) are irrelevant and set to zero. FIG. 1A shows normalized absorption and emission spectra of four spectrally overlapping classifier dyes (dye 1, dye 2, dye 4 and dye 4), overlaid with the channel-specific emission filters in the FACS CANTO II cytometer (c₁-c₄) used. For analyte detection, a blue-shifted reporter dye (R, BV-421) that does not interfere with barcode responses was selected. For instance, β₃₁=0 because dye 1 is not excited during the registration of intensity in c₃. FIG. 1B is a schematic representation of BMP readout by flow cytometry, indicating the lasers used for excitation and their corresponding channels. Note that any suitable light source, including lasers, may be used. Direct excitation of dyes as well as potential energy transfer pathways are highlighted in each flow cell to show the propensity for mFRET and mFRET cascades. In FIGS. 1C and 1D it is shown the effects of spectral overlap on the relative positions of BMP clusters given by their dye proportions (δ₁, δ₂, δ₃, δ₄) within the intensity spaces (FIG. 1C) I₁-I₂ and (FIG. 1D) I₃-I₄. Bleed-through is quantified by the fraction of dye f fluorescence leaking into channel c (βc_{f}). For example, BMPs (k, 0, 0, 0) and (-, -; k;0), where k is an arbitrary non-zero number and ‘-’ may take any value, would also be detected by c₂ (panel c) and c₄ (panel d) respectively. FRET, which is quantified by the efficiency of transfer (E_{da}) from donor d to acceptor a, occurs across all dyes in this setup and results in a strongly non-orthogonal response. Note that adding dye (2) to a BMP from (k, 0, 0, 0) to (k, k, 0, 0) in (c) can result in a decreased I₁ value due to E₁₂. Similarly, going from (k, k, 0, 0) to (k, k, k, k), a decrease in I₁ and I₂ is expected in (c) because the presence of dyes 3 and 4 at a significant density will lead to mFRET to these long-wavelengths dyes. There are 13 variables representing key physical parameters that need to be extracted, which are completed by measuring 18 judiciously selected barcodes. Briefly, single-color BMPs are measured to fit the values of μ_{f} and β_{cf} to the response using relative weighting, whereas two-color BMPs allow calculating the remaining variables.

### Example 8—Data Analysis

All data analysis was performed in MATLAB. To quantitatively compare fluorescence intensities across cytometry experiments, a linear normalization was performed on signal-to-background ratios to account for differences in laser power intensities. Single-bead were distinguished from bead aggregates and dust by using forward and side-scatter intensities and gating was automated using a MATLAB script for all data. Experimental emFRET was measured using the donor quenching method for select barcodes that allow crosstalk free measurement of a single donor species (e.g. I₁ when n₂=0 or vice versa). Therefore, by measuring donor-associated channel (c=d) for BMPs with and without any acceptor species (I_{c}^{FRET} and I_{c}^{noFRET} respectively), the experimental emFRET efficiency can then be calculated using equation (1) for c=d where it can be shown that:

$${( {E}_{d}^{T} )}^{exp} = \frac{{I}_{c}^{noFRET} - {I}_{c}^{FRET}}{{I}_{c}^{noFRET} - {I}_{c}^{0}} . \qquad ( 8 )$$

### Example 9—in Silico Design of Barcode Responses

The predicted BMP intensities were represented as regions that delimit a 35% variation from their center, a value that is ˜3:5× the measured standard deviation (see FIG. 4A) and thus expected to include >99% of the BMPs for a normal distribution. Regions in the I₃-I₄ joint intensity space were designed first as they are only dependent on (n₃, n₄).

28 non-overlapping regions were generated as shown in the bottom left plot of FIG. 4. Next, for each of the (n₃, n₄) values, regions of the I₁-I₂ intensity space were optimized and, as expected, were strongly dependent on (n₃, n₄) due to emFRET. Aside from a strong dependence on emFRET, the number of regions in I₁-I₂ was also limited by the requirement of a conserved total LO of 90 pmol (e.g. n₁+n₂≤30 pmol for barcodes (n₁, n₂, 5, 55)). The barcodes are represented and plotted as circular regions with a radius equal to a 35% variation to account for experimental variation of the BMP clusters. The dye proportions (n₁, n₂, n₃, n₄) for each barcode was chosen such that overlap between circles is avoided while occupying the entire spectral intensity space to increase the barcoding capacity. Graph at the lower left corner shows I₃-I₄ intensity space. Graphs in the central block show I₁-I₂ space for a set value of n₃ and n₄. In general, n₃ increases from left to right, and n₄ from bottom to top. The marginal plots in the bottom and to the left are the I₃-I₄ projections of the subsets plotted in the associated column and row, respectively. The range of barcode numbers for each subset is listed in the bottom right of each sub-plot.

In FIG. 4, it is shown the breakdown of inter-dye FRET efficiencies between all dye combinations for each barcode showcasing the extreme levels of FRET in some cases. In FIGS. 4-2E, the experimental intensity scatter plots of BMPs are overlaid with their MFM-predicted values from the three sub-plots highlighted in FIG. 4 to showcase the excellent agreement with the MFM, save some for barcodes with high n3 and n4 in FIG. 4.

### Example 10—Automated Decoding

To decode BMPs based on 4D intensity data (I₁, I₂, I₃, I₄), sequential 2D clustering, classification and assignment of BMPs of the pairwise channel intensities was performed. The BMP mixture was initially classified according to the I₃-I₄ intensity scatter plot, and each BMP classified to a cluster with an assigned (n₃, n₄) value. This was followed by independently decoding each of these clusters in the I₁-I₂ intensity space following the same protocol to complete the decoding of the (n₁, n₂, n₃, n₄) value. Gaussian-mixture model (GMM) was used to model a 2D intensity dataset, I, to the probability distribution function given by

$$p ( I ) = {\sum }_{k = 1}^{K} {π}_{k} 𝒩 ( I \vert {M}_{k} , {Σ}_{k} ) \qquad ( 9 )$$

where Mₖ, and Σₖ are, respectively, the means and covariances of the k Gaussian given by [symbol] (I|Mₖ,Σₖ), and πₖ are the mixing coefficients which a normalized metric that denotes how well the BMPs fit the k-th Gaussian. The total number of clusters, K, is defined by the number of unique combinations of dye proportions to be decoded in the corresponding space (e.g. number of unique (n₃, n₄) when classifying the (I₃, I₄) data). When using the MFM model, the expected intensities for every barcode are used as the initial value of the means in the GMM, that is, Mₖ⁰=I^{MFM}. Without the MFM, a set of arbitrary intensity values from the experimental dataset are used as the initial means. For both methods, the initial covariance matrix value was a diagonal matrix with 10% CV in each dimension (Σₖ⁰=0.1×Mₖ⁰), in accordance with the measured CV values in FIG. 2B, and the initial cluster probabilities to be homogeneous (πₖ⁰=1/K).

When I is an experimental dataset, p(I) is a measure of likelihood that this dataset is fit by the GMM clusters. During the expectation step of the expectation-maximization search, the probability that a certain BMP ψ, belongs to a cluster k, also referred to as the posterior probability, is calculated using:

$${φ}_{ψ k} = \frac{{π}_{k} 𝒩 ( {I}_{ψ} \vert {M}_{k} , {Σ}_{k} )}{p ( {I}_{ψ} )} . \qquad ( 10 )$$

During the maximization step, the values of the Gaussian components (Mₖ, Σₖ, and πₖ) are updated to maximize the log-likelihood (i.e. ln(p(I))). This process is repeated for up to 5000 iterations or until the condition for convergence (ln(p(I))<1e⁷) is reached. Typically, the GMM performs ‘soft classification’, whereby the W-th BMP is assigned to the population for which it has the maximal ϕ_{ψk}. To improve the fraction of correctly classified BMPs after the GMM converged to its solution, a posterior probability threshold was used, and varied from 50 to 100%, thereby rejecting BMPs with lower ϕ. Finally, to perform 2D cluster-to-barcode assignment without 5use of the MFM, and thus without a priori knowledge of the mean intensities of the barcode clusters, the means of the GMM-clusters intensities as well as the input dye proportions were sorted and assigned according to their root mean square (i.e. [Mₖ(1²+Mₖ(2)²]^{1/2} and [nₖ(1)²+nₖ(2)²]^{1/2}, respectively).

### Example 11—Calibration of the MFM to Extract Physical Parameters

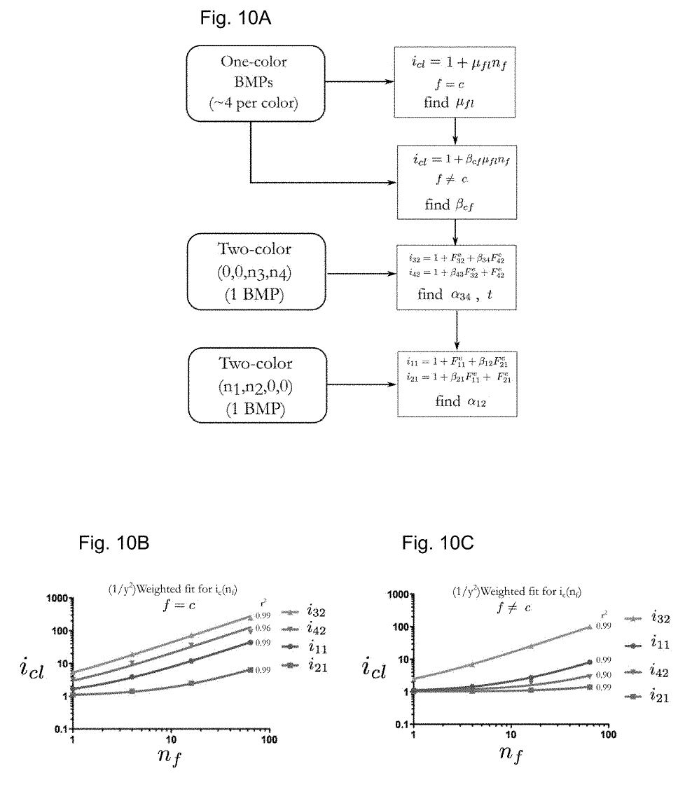

The parameters within the MFM equations were determined using 18 selected barcodes via the process flow described here (FIG. 10A). First, one-color BMPs are used to perform a linear fit of the linear basal fluorescence to input dye amounts using the equations shown, and extract direct excitation constants. Second, the same one-color BMPs barcodes can be fit to off detectors (f does not equal c) intensities as shown by the equations, to determine the bleed through constants. Finally, the FRET proportionality and labeling constants are determined using two-color BMPs. FIGS. 8B and 8C show fitting of the one-color BMPs by linear regression to calculate the (FIG. 10B) direct excitation and (FIG. 8C) bleed-through constants was performed using relative weighting (1/y2) during least-squares minimization which accounts for the heteroscedasticity of the BMP intensity where CV=cte with respect the dye concentrations (FIG. 4B). The fitted lines are plotted and the R-square values are presented next to each fit.

While the description has been described in connection with specific embodiments thereof, it will be understood that it is capable of further modifications and this application is intended to cover any variations, uses, or adaptations, including such departures from the present disclosure as come within known or customary practice within the art and as may be applied to the essential features hereinbefore set forth, and as follows in the scope of the appended claims.

What is claimed is:

1. A method for optimizing detection of a plurality of light-emissive components from a multi-fluorescence spectra, the method being executable by a processor of a computer system operatively communicating with an imaging device, the method comprising:
a) obtaining a multi-fluorescence based spectra of at least some of the light-emissive components;
b) determining a model of ensemble multi-fluorescence of the light-emissive components and of the imaging device, wherein the light-emissive components are stochastically distributed; and
c) determining proportion of each light-emissive component of the multi-fluorescence based spectra of a) based on the model of b).

2. The method as defined in claim 1, wherein the plurality of light-emissive components comprises at least four light-emissive components.

3. The method as defined in claim 1, wherein the imaging device comprises a plurality of detectors and the model of ensemble multi-fluorescence accounts for bleed-through between light-emissive components and the plurality of detectors.

4. The method as defined in claim 3, wherein the model of ensemble multi-fluorescence also accounts for multicolor fluorescence resonance energy transfer (mFRET) between the light-emissive components.

5. The method as defined in claim 4, wherein the model of ensemble multi-fluorescence also accounts for mFRET cascades between at least some of the light-emissive components.

6. The method as defined in claim 1, wherein the model of ensemble multi-fluorescence is based on an assumption that concentration of each of the light-emissive components is independent of one another.

7. The method as defined in claim 1, wherein the model of ensemble multi-fluorescence accounts for energy transfer between pairs of light-emissive components.

8. The method as defined in claim 4, wherein the accounting for mFRET between light-emissive components includes determining ensemble multicolor FRET efficiency (E^{T}_{d}) using the equation:
$${E}_{d}^{T} = \frac{{( {ω}_{d}^{T} / γ )}^{λ}}{1 + {( {ω}_{d}^{T} / γ )}^{λ}} ,$$
wherein ω_{d}^{T}, is a multicolor Förster acceptor number, and where γ and λ are exclusion and fitting constants, respectively.

9. The method as defined in claim 1, wherein at least some of the light-emissive components spectrally overlap.

10. The method as defined in claim 1, wherein a) is performed using the imaging device.

11. The method as defined in claim 1, wherein at least some of the light-emissive components are stochastically attached to particles.

12. The method as defined in claim 11, wherein the particles are microparticles

13. The method as defined in claim 1, wherein at least some of the light-emissive components are attached to a substrate.

14. A method for calibrating a multi-fluorescence model of a plurality of light-emissive components and an imaging device, the method being executable by a processor of a computer system operatively communicating with the imaging device, the method comprising:
a) obtaining a first fluorescence information about the individual light-emissive components using the imaging device;
b) obtaining a second fluorescence information about at least some pairs of light-emissive components using the imaging device; and
c) determining the constants of the multicolor fluorescence model using the first and second fluorescent information obtained in a) and b);
wherein at least some of the constants obtained in c) account for the non-linearity in the multicolor fluorescence model.

15. The method as defined in claim 14, wherein at least some of the light-emissive components are stochastically distributed.

16. The method as defined in claim 14, wherein the plurality of light-emissive components comprises at least four light-emissive components.

17. The method as defined in claim 15, wherein at least some of the emissive components are attached to particles.

18. The method as defined in claim 14, wherein the constants account for energy transfer between at least some of the light-emissive component pairs.

19. A method for optimizing proportions of a plurality of stochastically-attached light-emissive components across a set of particles,
a) obtaining a plurality of light-emissive components conjugated to a polymer cross-linker,
b) providing in solution a mixture containing a pre-determined proportion of the light-emissive component conjugated to polymer cross-linkers and unconjugated polymer cross-linker,
c) attaching the mixture in b) on microparticles by conjugating the polymer cross-linker to the particles
wherein the total number of polymer cross-linkers in b) remains constant across the sets of particles.


SEQUENCE LISTING (2 sequences)

SEQ ID NO: 1 - DNA, 21 bp, Artificial Sequence
  Synthetic oligonucleotide
tttttttttgtggcggcggtg

SEQ ID NO: 2 - DNA, 21 bp, Artificial Sequence
  Synthetic oligonucleotide
caccgccgccacaaaaaaaaa